(19) United States
(12) Patent Application Publication
(10) Pub. No.: US 2010/0185219 A1
(43) Pub. Date: Jul. 22, 2010

(54) REINFORCED BIOLOGICAL MESH FOR SURGICAL REINFORCEMENT

(75) Inventors: Arthur A. Gertzman, Flemington, NJ (US); Michael Schuler, Princeton, NJ (US)

Correspondence Address: FROMMER LAWRENCE & HAUG, 745 FIFTH AVENUE- 10TH FL., NEW YORK, NY, 10151, US

(73) Assignee: Musculosketetal Transplant Foundation, Edison, NJ (US)

(21) Appl. No.: 12/597,672

(22) PCT Filed: Apr. 25, 2008
(86) PCT No.: PCT/US08/61618

§ 371 Date: Mar. 29, 2010

Related U.S. Application Data
(60) Provisional application No. 60/907,979, filed on Apr. 25, 2007.
(60) Provisional application No. 60/929,186, filed on Jun. 18, 2007.

(51) Int. Cl. A61F 2/00; B32B 37/00; B32B 37/12; B23P 11/00; A61K 38/57; A61K 38/06; A61K 38/08

(52) U.S. Cl. 606/151; 156/60; 156/325; 29/525.01; 514/12; 514/18; 514/17

(57) ABSTRACT

The invention is directed toward a composite material for use in a medical application, comprising a biological material and a reinforcement material. The biological material may be overlayed onto the reinforcement layer, or the material may be attached together. In one embodiment, the composite material may be arranged in layers, such that the biological material is in a first layer and the reinforcement material is in a second layer. In another embodiment, the reinforcement material may be in a layer sandwiched between two layers of biological material. In a certain embodiment, the reinforcement material is in the form of a mesh.

### INCORPORATION BY REFERENCE

This application claims the benefit of priority of U.S. Provisional Application No. 60/907,979 filed Apr. 25, 2007, and of U.S. Provisional Application No. 60/929,084 filed Jun. 12, 2007.

The foregoing applications, and all documents cited therein or during their prosecution (“application cited documents”) and all documents cited or referenced in the application cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention.

### FIELD OF THE INVENTION

The present invention is generally directed toward an implantable reinforced biological prosthesis and in certain embodiments is directed toward a resilient, bioremodelable, biocompatible soft tissue provided with a mesh reinforcement which is used to repair, augment, or replace human tissue. The prosthesis acts as a template by which the host's tissues will remodel through a process that will replace the prosthesis molecules with the appropriate host cells in order to restore and replace the original host tissue while the mesh reinforcement adds structural strength to the prosthesis.

### BACKGROUND OF THE INVENTION

Despite the growing sophistication of medical technology, repairing and replacing damaged tissues remains a frequent, costly, and serious problem in health care. Currently implantable prostheses are primarily made from a number of synthetic and treated natural materials. In reinforcing or repairing hernias and abdominal wall defects, several prosthetic materials have been used, including tantalum gauze, stainless mesh, DACRON7, ORLON7, FORTISAN7, nylon, knitted polypropylene (MARLEX7), microporous expanded-polytetrafluoroethylene (GORE-TEXT), dacron reinforced silicone rubber (SILASTIC7), polyglactin 910 (VICRYL7), polyester (MERSILENE7), polyglycolic acid (DEXON7), processed sheep dermal collagen (PSDC7) crosslinked bovine pericardium (PERI-GUARD7), laminated sheet intestinal submucosa (RESTORESIS7), and preserved human dura (LYODURA7). No single prosthetic material has gained universal acceptance.

The major advantage of metallic meshes is that they are inert, resistant to infection and can stimulate fibroplasia. Their major disadvantage is the fragmentation that occurs after the first year of implantation as well as the lack of malleability. Synthetic meshes have the advantage of being easily molded and, except for nylon, retain their tensile strength in the body. European Patent No. 91122196.8 to Krajicek details a triple-layer vascular prosthesis which utilizes non-resorbable, synthetic mesh as the center layer. The synthetic textile mesh layer is used as a central frame to which layers of collagenous fibers can be added, resulting in the tri-layered prosthetic device. The major disadvantage of a non-resorbable synthetic mesh is lack of inertness, susceptibility to infection, and interference with wound healing.

Absorbable synthetic meshes often have the disadvantage of losing their mechanical strength, because of dissolution by the host, prior to adequate cell and tissue ingrowth. A widely used material for abdominal wall replacement and for reinforcement during hernia repairs is MARLEX7, a polypropylene mesh graft. However, such grafts have been reported to cause moderate to severe adhesions. GORE-TEX7 is probably the most chemically inert polymer and has been found to cause minimal foreign body reaction when implanted. A major problem exists with the use of polytetrafluoroethylene in a contaminated wound as it does not allow for any macromolecular drainage, which limits treatment of infections. Meshes constructed of 100% synthetic fiber are not recommended because they can interact with the underlying tissue (periosteum or intestine, in the case of abdominal hernia) and adhere to these tissues which interfere with the functions of these tissues. Collagen first gained utility as a material for medical use because it was a natural biological prosthetic substitute that was in abundant supply from various animal sources. The objectives for the original collagen prosthetics were that the prosthesis should continue to provide strength and essentially act as an inert material. With these objectives in mind, purification and crosslinking methods using crosslinking agents including glutaraldehyde, formaldehyde, polyepoxides, and diisocyanates were developed to enhance mechanical strength and decrease the degradation rate of the collagen. In general, these crosslinking agents generated collagenous material which resembled a synthetic material more than a natural biological tissue, both mechanically and biologically. Crosslinking native collagen reduces the antigenicity of the material by linking the antigenic epitopes rendering them either inaccessible to phagocytosis or unrecognizable by the immune system.

All of the above problems associated with traditional materials stem, in part, from the inability of the body to recognize an implant as “inert”. When a prosthesis is implanted, it should immediately serve its requisite mechanical and/or biological function as a body part. The prosthesis should also support appropriate host cellularization by ingrowth of mesenchymal cells, and in time, be replaced with host tissue. In order to do this, the implant must not elicit a significant immune response or be either cytotoxic or pyrogenic to promote healing and development of the neo-tissue. Prostheses or prosthetic material derived from explanted mammalian tissue have been widely investigated for surgical repair or for tissue and organ replacement. The tissue is typically processed to remove cellular components leaving a natural acellular tissue matrix.

U.S. Pat. No. 3,562,820 issued Feb. 16, 1971 discloses tubular, sheet, and strip forms of prostheses formed from submucosa adhered together by use of a binder paste such as a collagen fiber paste or by use of an acid or alkaline medium.

U.S. Pat. No. 4,502,159 issued Mar. 5, 1988 discloses a tubular prosthesis formed from pericardial tissue in which the tissue is cleaned of fat, fibers and extraneous debris and then placed in phosphate buffered saline. The pericardial tissue is then placed on a mandrel and the seam is then closed by suture and the tissue is then crosslinked.

U.S. Pat. No. 4,801,299 issued Jan. 31, 1989 discloses a method of processing body derived structures for implantation by treating the body derived tissue with detergents to remove cellular structures, nucleic acids, and lipids, to leave an extracellular matrix which is then sterilized before implantation.

U.S. Pat. No. 7,070,558 issued Jul. 4, 2006 discloses a sling having two rectangular sheets of mammalian tissue sandwiching mesh, weave or braid made from material such as nylon, polyethylene, polyester, polypropylene, fluoropolymers or other suitable synthetic materials.

It is a continuing goal to develop implantable prostheses which can successfully be used to replace or to facilitate the repair of human tissues, such as hernias, abdominal wall defects, and mammary skin so that the intrinsic strength, resilience, and biocompatability of the host's own cells may be optimally exploited in the repair process. In around 40% to 50% of medical cases, when the skin remodels, the implant is replaced with weaker tissue. Another problem with the use of biological meshes in hernia applications is the tendency of bacteria to cause the implant to be absorbed. The bacteria excrete protease enzymes which chemically react with the collagen in the matrix and cause it to break down and eventually resorb.

### SUMMARY OF THE INVENTION

The instant invention relates to a composite material for use in a medical application, comprising at least one biological material and at least one reinforcement material. In certain embodiments, the biological material partially overlays the reinforcement material, while in other embodiments, the biological material overlays substantially all of the reinforcement material.

In certain embodiments, the biological material is attached to the reinforcement material. In some embodiments the biological material is attached to the reinforcement material via an adhesive. Suitable examples of adhesives include cyanoacrylate, glue, fibrin glue, fibrin, thrombin, plasma, and cellular derived hemostatic agents. In other embodiments the biological material is attached via a mechanical agent; suitable examples of mechanical agents include sutures or staples. In yet other embodiments, fibers of the biological material are interwoven with fibers of the reinforcement material. Also, in certain embodiments, the biological material and the reinforcement material are attached through physical or chemical crosslinking. Suitable examples of physical crosslinking are dehydrothermal crosslinking, ultraviolet light, and heat, while suitable examples of chemical crosslinking are glutaraldehyde, formaldehyde, and carbodiimide. In other embodiments, the biological material and the reinforcement material may be attached by swelling the reinforcement material or creating cavities within the reinforcement material, and then placing or precipitating the biological material into the reinforcement material. In yet other embodiments, the reinforcement material may be coated or sprayed with the biological material.

In certain embodiments, the biological material is in a first layer, and the reinforcement material is in an adjacent second layer. In some embodiments, the biological material is further in a third layer adjacent to the reinforcement material, such that the second layer of reinforcement material is between the first layer of biological material and the third layer of biological material; in some embodiments, the biological material of the first layer is the same as the biological material of the third layer, while in other embodiments, the biological material of the first layer is different than the biological material of the third layer.

In some embodiments, the reinforcement material is in the form of a mesh. In certain embodiments, the mesh comprises a web, such that the web is defined by a plurality of spaced apertures. A suitable example of the size of the spaced apertures is about 0.1 cm to about 2.0 cm.

In certain embodiments of the invention, the biological material may be allograft, xenograft, autograft, or biologic matrix. In. some embodiments, the biological material is acellular. In certain embodiments, the allograft, xenograft, or autograft is dermis, fascia, fascia lata tendon, pericardia, ligament, or muscle.

In some embodiments of the invention, the reinforcement material is non-biologic. Suitable examples of non-biologic reinforcement material in certain embodiments include non-absorbable fibers consisting of nylon, polyester, polypropylene, silk and cotton. In some embodiments of the invention, the non-biologic reinforcement material is multifilament polyester strands, while in other embodiments, the non-biologic reinforcement material is monofilament strands.

In certain embodiments of the invention, the reinforcement material is biologic. In some embodiments, the biologic reinforcement material is selected from the group consisting of allograft, xenograft, autograft, and biologic matrix. In other embodiments, the biologic reinforcement material is extracellular matrix proteins. Suitable examples of extracellular matrix proteins in certain embodiments are collagen, elastin, hyaluronic acid, and glycosaminoglycans. In some embodiments, the biologic reinforcement material is connective tissue. Suitable examples of connective tissue include tendon, ligament, and fascia. In yet other embodiments, the biologic reinforcement material is bone or muscle. In certain embodiments of the invention, the reinforcement material can sustain a load of at least 10 Newtons.

The instant invention also relates to a method of preparing a composite material for use in a medical application, comprising providing at least one biological material and at least one reinforcement material, and either overlaying the reinforcement material with the biological material, or attaching the biological material to the reinforcement material.

In certain embodiments of the methods of the invention, the biological material is attached to the reinforcement material via an adhesive; suitable examples of an adhesive include cyanoacrylate, glue, fibrin glue, fibrin, thrombin, plasma, and cellular-derived hemostatic agents. In other examples of the invention, the biological material is attached to the reinforcement material via a mechanical agent; suitable examples of mechanical agents include sutures and staples. In yet other embodiments of the methods of the invention, fibers of the biological material are interwoven with fibers of the reinforcement material. Also, in certain embodiments, the biological material and the reinforcement material are attached through physical or chemical crosslinking. Suitable examples of physical crosslinking arc dehydrothermal crosslinking, ultraviolet light, and heat, while suitable examples of chemical crosslinking are glutaraldehyde, formaldehyde, and carbodiimide. In yet other embodiments, the biological material and the reinforcement material may be attached by swelling the reinforcement material or creating cavities within the reinforcement material, and then placing or precipitating the biological material into the reinforcement material. In another embodiment, the reinforcement material may be coated or sprayed with the biological material.

In further embodiments of the methods of the invention, the biological material is selected from the group consisting of allograft, xenograft, autograft, and biologic matrix. In some embodiments, the biological material is acellular. In certain embodiments, the allograft, xenograft, or autograft is selected from the group consisting of dermis, fascia, fascia lata tendon, pericardia, ligament, and muscle.

In some embodiments of methods of the invention, the reinforcement material is non-biologic. Suitable examples of non-biologic reinforcement material in some embodiments include non-absorbable fibers consisting of nylon, polyester, polypropylene, silk and cotton. In certain embodiments, the non-biologic reinforcement material is multifilament polyester strands. In other embodiments, the non-biologic reinforcement material is monofilament strands.

In yet further embodiments of the instant invention, the reinforcement material is biologic. In certain embodiments, the biologic reinforcement material is selected from the group consisting of allograft, xenograft, autograft, and biologic matrix. In some embodiments, the biologic reinforcement material is extracellular matrix (ECM) proteins. In embodiments of the invention, the biologic reinforcement material is provided by precipitation of a particulate composition of ECM proteins. In other embodiments, the biologic reinforcement material is provided by linking ECM proteins together to form larger molecules. Suitable examples of extracellular matrix proteins are collagen, elastin, hyaluronic acid, and glycosaminoglycans. In some embodiments, the biologic reinforcement material is connective tissue; suitable examples include tendon, ligament, and fascia. In other embodiments, the biologic reinforcement material is bone or muscle.

In some embodiments of the invention, the biologic reinforcement material is provided by electrospinning biologic fibers. In other embodiments, the biologic reinforcement material is provided by extruding biologic fibers. In yet other embodiments, the biologic reinforcement material is provided by attaching nanoparticles to create larger ECM-based molecules, which forms the reinforcement material. In further embodiments, the biologic reinforcement material is provided by using recombinant viral DNA to produce matrix from biologic material.

In some embodiments, the methods of the invention further comprise treating the biological material with at least one growth factor; suitable examples of growth factors in some embodiments include platelet-derived growth factor (PDGF), fibroblast growth factor (FGF 1-23) and variants thereof, transforming growth factor-beta (TGF-beta) and vascular endothelium growth factor (VEGF), Activin/TGF, steroids, or any combination thereof. In certain embodiments, the methods of the invention further comprise treating the reinforcement material with at least one anti-infectant; suitable examples of the anti-infectant are anti-inflammatory agents, analgesic agents, local anesthetic agents, antispasmodic agents, or combinations thereof. In further embodiments, the methods of the invention additionally comprise treating the composite material with one or more protease inhibitors; suitable examples of protease inhibitors include Aminoethylbenzenesulfonyl fluoride HCL, Aprotinin, Protease Inhibitor E-64, Leupeptin, Hemisulfate, EDTA, Disodium (0.025-0.10 um) and trypsin-like proteases, Pepstatin A (Aspartic Proteases), Marmistat (MMP2), or any combination thereof.

Finally, the instant invention relates to a method of repairing damaged tissue, comprising implanting the composite material into the site of the damaged tissue.

These and other embodiments are disclosed or are obvious from and encompassed by, the following Detailed Description.

### BRIEF DESCRIPTION OF THE DRAWINGS

The following detailed description, given by way of example, but not intended to limit the invention solely to the specific embodiments described, may best be understood in conjunction with the accompanying drawings, in which:

FIG. 1 is a plan view of the implant invention with a mesh reinforcement of spaced fused multifilament polyester strands;

FIG. 2 is a plan view of the reinforced implant with a mesh reinforcement of monofilament strands held in place by sutures;

FIG. 3 is a plan view of the reinforced implant with tendon/ligament reinforcement strands held in place by sutures; and

FIG. 4 is an enlarged cross sectional view of another embodiment of the invention showing reinforcing mesh within two dermal layers positioned on each side of the reinforcing middle mesh.

### DESCRIPTION OF THE INVENTION

Described herein is a composite material for use in a medical application comprising a biological material and a reinforcement material, a method of preparing the composite material, and a method of repairing damaged tissue using the composite material.

### Biological Material

The biological material of the instant invention may generally serve as a temporary tissue substitute and template for new tissue formation. It also may support appropriate host cellularization by ingrowth of blood vessels and cells such as inflammatory cells, mesenchymal cells, fibroblasts and other cells, which may be necessary in order for the biological material to be eventually replaced by host tissue.

The biological material used herein may include any material derived from a living or once-living source. Importantly, these may include allograft, xenograft, and autograft tissues (collectively referred to herein as “grafts”), as well as biologic matrices derived from tissue sources.

The term “allograft” refers to a transplant comprising cells, tissues, or organs sourced from another member of the same species. The member of the same species may be living or nonliving.

The term “xenograft” refers to a transplant comprising cells, tissues, or organs sourced from another species. Examples of species that commonly serve as a xenograft source include, but are not limited to, simian, porcine, bovine, ovine, equine, feline, and canine.

Finally, the term “autograft” refers to cells, tissues, or organs transplanted from one site to another on the same patient.

Examples of tissues that are typically used as an allograft, xenograft, or autograft may include, but are not limited to, musculoskeletal tissues such as bone and muscle; cardiovascular tissue such as heart valves and blood vessels, connective tissue such as ligaments, tendons, fascia, and cartilage; dermal tissue such as dermis, epidermis, and whole skin; and neural tissue.

Alternatively, the biological tissue may be a biologic matrix derived from any number of tissue sources, in particular soft tissue sources, including dermal, fascia, dura, pericardia, tendons, ligaments, or muscle. The biologic matrix may comprise at least one anti-infective, preferably at least one slowed release anti-infective. Suitable dermal matrices include, for example, acellular dermal matrices such as the human acellular dermal matrices from the Flex HD® product line (available from Musculoskeletal Transplant Foundation, Edison, N.J.).

In certain embodiments, biological material of the present invention is taken from the dermis, fascia, fascia lata, pericardium, tendon, or ligament.

In another embodiment, the biological material may be acellular. The term “acellular” as used herein refers to lacking substantially all viable cells, including materials in which the concentration of viable cells is less than about 1% (e.g., less than 0.1%, 0.01%, 0.001%, 0.0001%, 0.00001%, or 0.000001%) of that in the tissue or organ from which the biological material was derived. An acellular biological material may also include materials comprising, after decellularization, about 25% or less of nucleic acid (e.g., DNA) that is present in normal cellularized biological materials. Examples of acellular biological material may include, but are not limited to, intact basement membrane or acellular musculoskeletal, cardiovascular, connective, dermal, and neural tissues. The decellularization may be achieved using methods known in the art, for example, by processing with 1 M NaCl and 0.1% of Trinton X-100.

In an alternative embodiment, the biological material may be a combination of cellular and acellular tissue.

The size and shape of the biological material may vary according to the medical application, and can be determined by one skilled in the art. For example, the shape of the biological material may be polygonal (triangular, rectangular, pentagonal, etc.), circular, or oval.

### Reinforcement Material

The reinforcement material of the instant invention may generally serve to provide strength and structural integrity to the biological tissue during its use in medical applications. The reinforcement material may typically support the biological tissue and the surrounding tissue in general during wound repair and tissue closure.

In certain embodiments, the reinforcement material may be biocompatible. As used herein, the term “biocompatible” refers to a material that is substantially non-toxic and that does not induce a significantly adverse effect on the patient's health and may be biodegradable.

Selection of the reinforcement material may take into consideration the pore size, strength, permeability and flexibility of the material, as well as the structure and function of the surrounding tissue. For example, for use in applications involving load-bearing tissue, reinforcement materials may provide the appropriate tensile strength and flexibility to support the biological material and surrounding tissue during the formation of new tissue sufficient to support surrounding tissue. One of ordinary skill in the art can recognize the desired characteristics of the reinforcement material in selecting the optimal material.

Furthermore, the reinforcement materials may be absorbable or non-absorbable. Absorbable materials allow for the tissue being supported to properly heal, although the degradation rate of the reinforcement material is preferably slower than the degradation rate of the biological material. Non-absorbable fibers may be used, for example, in diabetic or diet deficient patients where the tissue and mesh absorbs rapidly. One skilled in the art can readily determine if and when absorbable or non-absorbable reinforcement materials should be used.

The reinforcement material may be non-biologic, biologic, or a combination of both. Examples of non-biologic reinforcement materials may include, but are not limited to, polypropylene mesh such as Prolene™ (Ethicon Inc., Somerville, N.J.) and Marlex™ (C. R. Bard Inc.); polyester such as Dacron™ and Mersilene™ (Ethicon Inc., Somerville, N.J.); silicone, polyethylene, polyamide, titanium, stainless steel, polymethylmethacrylate, nylon, silk, cotton; polyglactic acid such as Vicryl™ mesh (Ethicon Inc., Somerville, N.J.), polyglycolic acid such as Dexon™ mesh; poliglecaprone, collagen, polydioxone and expanded polytetrafluoroethylene such as DualMesh™, Mycromesh™, or other expanded PTFE (W. L. Gore and Associates); PDS®, Vicryl®, or Monocryl®. In one embodiment, the reinforcement material may be multifilament polyester strands or monofilament polyester strands.

Biologic reinforcement material as used herein may include any material derived from a living or once-living source, which includes allograft, xenograft, and autograft tissues, and biologic matrices derived from tissue sources. Examples of tissues that are typically used as an allograft, xenograft, or autograft may include, but are not limited to, musculoskeletal tissues such as bone grafts, and muscle; cardiovascular tissue such as heart valves and blood vessels, connective tissue such as ligaments, tendons, fascia, and cartilage; dermal tissue such as dermis, epidermis, and whole skin; and neural tissue. In particular embodiments, the biologic reinforcement material is tendon, ligament, or fascia.

Biologic matrix may be derived from any number of tissue sources, in particular soft tissue sources, including dermal, fascia, dura, pericardia, tendons, ligaments, or muscle. The biologic matrix may comprise at least one anti-infective, and preferably'at least one slowed release anti-infective. Suitable dermal matrices include, for example, acellular dermal matrices such as the human acellular dermal matrices from the Flex HD® product line (available from Musculoskeletal Transplant Foundation, Edison, N.J.).

In another embodiment, the biologic reinforcement material may be acellular, such as intact basement membrane or acellular musculoskeletal, cardiovascular, connective, dermal, or neural tissues. The decellularization may be achieved using methods known in the art, for example, by processing with 1 M NaCl and 0.1% of Trinton X-100.

In an alternative embodiment, the biologic reinforcement material may be a combination of cellular and acellular tissue.

In yet another embodiment, the biologic reinforcement material may be extracellular matrix protein such as, but not limited to, collagen, elastin, hyaluronic acid, or glycosaminoglycans.

In one embodiment, the reinforcement material may undergo a crosslinking treatment to alter the mechanical properties of the material. For example, the reinforcement material may undergo crosslinking treatment to increase the strength of the material for medical applications in load-bearing tissue.

The reinforcement material may be any shape or size according to its application as a support to the biological material in medical applications. Selection of the appropriate shape or size of the reinforcement material is routine for one of ordinary skill in the art. For example, the reinforcement material may be in the form of fibers organized as a mesh or lattice. In one embodiment, the mesh may be comprised of a web, wherein the web is defined by a plurality of spaced apertures. The mesh or lattice can have various designs such as polygons (triangles, rectangles, etc.), circles, ovals, spirals, or any combination thereof. The spaces between the fibers of the mesh can vary according to the size of the mesh and the medical application (e.g., for implantation in a load-bearing tissue), but are preferably between about 0.1 cm and about 2.0 cm.

### Composite Material Structure

The composite material of the invention is comprised of at least one biological material and at least one reinforcement material. The structure and arrangement of the composite structure will depend upon its intended medical application. For instance, the composite material may be in the shape of a rectangular sheet if it is to be used to repair hernias or abdominal wall defects. One skilled in the art can determine the optimal shape of composite material based on its intended application.

The composite material may contain particular mechanical properties which make it ideal for implantation. These properties can be determined by one skilled in the art. For example, the composite material may be designed to sustain a load of at least about 10 Newtons.

In one embodiment, the composite material may be comprised of a first and second layer, such that the first layer is comprised of a biological material and the second layer is comprised of a reinforcement material. The biological material layer and the reinforcement layer may be the same size or a different size; for example, the reinforcement material layer may be smaller than the biological material layer, if support of the entire biological material layer is unnecessary.

In another embodiment, the composite material may be comprised of three layers—a first and third outer layer, and a second inner layer—wherein the outside layers are comprised of a biological material and the inside layer is comprised of a reinforcement material. The outer biological material layer and the inner reinforcement layer may be the same size or a different size. The biological material of the first layer may be the same as the biological material of the third layer, or the biological material of the first layer may be different than the biological material of the third layer. In an alternative embodiment, the outer layers are comprised of reinforcement material and the inner layer is comprised of a biological tissue.

The composite material may also be substantially in the shape of a tube. In one embodiment, the substantially tubular composite material may comprise outer and inner concentric layers, such that the outer layer comprises the biological material and the inner layer comprises the reinforcement material, or vice versa. Alternatively, the substantially tubular composite material may comprise two adjacent layers which spiral together from the center of the tube, wherein the outermost layer comprises the biological material and the innermost layer comprises the reinforcement material, or vice versa. In yet another embodiment, the substantially tubular composite material may comprise a biological material and a reinforcement material which intertwine together as a double helix.

### Preparation of the Composite Material

The present invention relates to a method of preparing the composite material described herein. The method comprises providing at least one biological material and at least one reinforcement material, and then overlaying the reinforcement material with the biological material, or attaching the biological material to the reinforcement material.

As described above, the biological tissue may be any material derived from a living or once-living source, and includes allograft, xenograft, and autograft tissues, as well as biologic matrices derived from tissue sources. The graft tissues can be removed from living or once-living sources by methods known in the art, including standard surgical techniques or, in the case of dermal grafts, using a dermatome. The biological material may also be processed (e.g., decellularized, removal of unwanted materials) and shaped to the form that is appropriate for implantation using techniques known in the art. For example, the biological material can be decellularized using physical means, chemical methods (e.g., alkaline and acid treatments, non-ionic, ionic, and zwitterionic detergents, hypotonic and hypertonic treatments, chelating agents), enzymatic methods, protease inhibitors, and antibiotics (see Gilbert et al. “Decellularization of tissues and organs” Biomaterials 27(19): 3675-3683, 2006; incorporated by reference). Unwanted materials can be removed from the biological material through application of solutions comprising peracetic acid, povidone-iodine, or mixtures of antibiotics, or of gamma irradiation. Alternatively, a novel technique involving application of an ultrashort pulse laser may be employed to remove unwanted material and shape the biological tissue. This technique can precision ablate unwanted material from the surface of the biological tissue, and shape biological material by making precision cuts and section the material without damaging surrounding tissue.

As described previously, the reinforcement material may be non-biologic or biologic. Non-biologic reinforcement materials can be acquired from any commercial source and manipulated into the desired shape or form using techniques known in the art. For example, in forming the shape of a mesh, the reinforcement material can be an over- and underweave that is heat tacked at each junction point.

In embodiments wherein the reinforcement material is a biological material, the reinforcement material can be acquired via the same methods as described herein for the biological material. Other methods include precipitation of particulate composition of ECM proteins, extraction of ECM proteins from tissue via methods known in the art (e.g., see Lee “Protein extraction from mammalian tissues” Methods in Molecular Biology 362: 385-9, 2007; Bishop et al. “Extraction and characterization of the tissue forms of collagen types II and IX from bovine vitreous.” Biochemical Journal 299(Pt 2): 497-505, 1994; Rajan et al. “Preparation of ready-to-use, storable and reconstituted type I collagen from rat tail tendon for tissue engineering applications” Nature Protocols 1(6): 2753-8, 2006; all incorporated by reference); attachment of nanoparticles to create larger ECM-based molecules, and attachment of ECM proteins linked together to create larger molecules. The reinforcement material may also be formed by production of ECM proteins such as collagen, elastin, hyaluronic acid, or GAGs using recombinant DNA.

In addition, the reinforcement material may be formed by electrospinning fibers comprising ECM proteins (see, for example, Li et al. “Electrospun protein fibers as matrices for tissue engineering” Biomaterials 26(30): 5999-6008, 2005; U.S. Pat. No. 6,790,455 to Chu et al.; all incorporated by reference) or by extruding ECM proteins (see, for example; Kato et al. “Formation of continuous collagen fibers: evaluation of biocompatibility and mechanical properties” Biomaterials 11: 169-75, 1990; Kato et al. “Mechanical properties of collagen fibers: a comparison of reconstituted rat tendon fibers” Biomaterials 10:38-42, 1989; U.S. Pat. No. 5,378,469 to Kemp, et al.; and U.S. Pat. No. 5,256,418 to Kemp, et al.; all incorporated by reference).

In some embodiments, once the biological material and the reinforcement material are provided, the biological material may overlay, either partially or substantially all, of the reinforcement material. In certain embodiments, the biological material is overlayed on the reinforcement material without any type of attachment.

In another embodiment, the biological material and the reinforcement material are attached together. For example, the materials may be attached using an adhesive such as, but not limited to, cyanoacrylate, glue, fibrin glue, fibrin, thrombin, plasma, or cellular-derived hemostatic agents. Alternatively, the biological material and the reinforcement material may be attached by using a mechanical agent such as a suture or a staple, or by interweaving fibers of the biological material with fibers of the reinforcement material.

In another embodiment, the biological material is attached to the reinforcement material by crosslinking. For instance, the materials may be attached using a physical crosslinking, such as ultraviolet radiation, dehydrothermal treatment, or heat, which are all known in the art (e.g., see Weadock et al. “Physical crosslinking of collagen fibers: comparison of ultraviolet irradiation and dehydrothermal treatment” Journal of Biomedical Materials Research 29(11): 1373-1379, 1995; incorporated by reference). Alternatively, the biological material and reinforcement material may be attached by chemical crosslinking using agents such as formaldehyde, glutaraldehyde, divinyl sulfone, a polyanhydride, a polyaldehyde, a polyhydric alcohol, carbodiimide, epichlorohydrin, ethylene glycol diglycidylether, butanediol diglycidylether, polyglycerol polyglycidylether, polyethylene glycol diglycidylether, polypropylene glycol diglycidylether, or a bis-or poly-epoxy cross-linker such as 1,2,3,4-diepoxybutane or 1,2,7,8-diepoxyoctane, which are all well-known in the art.

In another embodiment, the biological material may be attached to the reinforcement material by swelling the reinforcement material or creating cavities within the reinforcement material, and then placing or precipitating the biological material into the reinforcement material. In another embodiment, the reinforcement material may be coated or sprayed with the biological material. These techniques are well-known and can be conducted by one of ordinary skill in the art.

### Treatments of the Composite Material

During or after preparation of the composite material, the components of the composite material or the composite material itself may undergo certain treatments in order to have desired properties.

For instance, in one embodiment, the biological material may be treated with a growth factor such as, but not limited to, platelet-derived growth factor (PDGF), fibroblast growth factor (FGF 1-23) or variants thereof, transforming growth factor-beta (TGF-beta) or vascular endothelium growth factor (VEGF), Activin/TGF, steroids, or any combination thereof. The biological material may also be treated with a hormone such as estrogen, steroid hormones, or other hormones to promote growth of appropriate tissue, or stem cells or other suitable cells derived from the host patient, such as fibroblast, myoblast, or other progenitor cells to mature into appropriate tissues.

In another embodiment, the reinforcement material may be treated with an anti-infective agent. The addition of suitable anti-infective compounds to the surface of the mesh on the strands and junction points attack the bacteria and materials present from local infection or inhibit the growth and proliferation of these bacteria on and near the implant. Thus, while not wishing to be bound by theory, in addition to assisting in the management of the infections per se, it is believed that the anti-infective will delay the absorption of the biological tissue which will allow the implant to function longer as a supporting, load sharing scaffold in the surgical site and permit the patient's repair processes to remodel and achieve a stronger repair tissue. Examples of anti-infective agents include, but are not limited to anti-inflammatory agents, analgesic agents, local anesthetic agents, antispasmodic agents, or combinations thereof.

The reinforcement material may also be treated with a protease inhibitor in order to alter its degradation rate. Examples of protease inhibitors that can be used in this invention include, but are not limited to, Aminoethylbenzenesulfonyl fluoride HCL, Aprotinin, Protease Inhibitor E-64, Leupeptin, Hemisulfate, EDTA, Disodium (0.025-0.10 um) or trypsin-like proteases, Pepstatin A (Aspartic Proteases), Mannistat (MMP2), or any combination thereof.

All of these treatments described herein may be applied by methods known in the art, including, but not limited to, bathing, injecting, transfecting, bonding, coating, adding genetically modified cells and/or genetic material itself, or laminating.

In another embodiment, the biological material, the reinforcement material, or the composite structure as a whole may undergo a crosslinking treatment in order to alter and create distinctive mechanical properties for the components. The application of crosslinking treatments to alter mechanical properties is well-known in the art (e.g., see U.S. Pat. No. 6,184,266 to Ronan et al.; Elbjeirami et al. “Enhancing mechanical properties of tissue-engineered constructs via lysyl oxidase crosslinking activity” Journal of Biomedical Materials Research A 66(3): 513-521, 2003; all incorporated by reference).

### Medical Applications

The composite material of the present invention can be used to repair, augment, or replace human tissue, particularly in a wound or tissue defect. Examples of these applications include, but are not limited to, skin lesions, burns, traumatic wounds, hernias, abdominal defects, chest wall defects, cranial defects, pelvic defects, joint defects, and congenital abnormalities.

In one embodiment, the composite material can be used to repair wounds or defects of the skin, such as in burned patients, or patients undergoing reconstructive surgery, tissue trauma, surgical resection, infection, chronic skin diseases or chronic wounds. In another embodiment, the present invention may be used in the replacement of other specialized epithelial tissues in a variety of organ systems, including but not limited to, bone, cartilage, oral mucosa, uroepithelial, gastrointestinal, respiratory or vascular. The composite material of the present invention may also be used to replace tissue defects with a tissue composed of organ-specific cells identical to the native tissue, without having to disrupt uninjured organs for donor tissue. Such tissue can be replaced after surgical resection for malignancy, disease or trauma. Moreover, the composite material can be used to replace various commonly lost tissues such as oropharyngeal, nasal and bronchial mucosa, lip vermillion, blood vessels, trachea, esophagus, stomach, small and large bowel, biliary ducts, ureter, bladder, urethra, periosteum, synovium, areolar tissue, chest wall, abdominal wall or vaginal mucosa. Structural defects such as ventral, inguinal and diaphragmatic hernias, replacement or augmentation of tendons, ligaments or bone or abdominal or thoracic wall reconstruction can also be repaired as described herein. One of skill in the art can recognize alternative and various types of wounds or tissue defects for which the present compositions and methods will be useful.

In one embodiment, abnormal tissue may be intentionally (e.g., surgically) removed from an individual and new tissue can be elicited in its place by implantation of the composite material of the invention. Alternatively, composite material may be used to produce new tissue in place of tissue which has been lost due to accident or disease.

Before the composite material of the invention can be implanted, the wound or tissue requiring repair may be prepared. Any damaged of destroyed tissue may be surgically removed to prevent them from interfering with the healing process. Preferably, only intact cells are present at the perimeter of the wound or tissue.

The composite material may be implanted according to methods known in the art. For example, in one embodiment, the composite material may be draped across the wound with care taken to avoid the entrapment of air pockets between the wound or tissue and the composite material. The composite material may be sutured or stapled to the wound or tissue using conventional techniques and the wound or tissue is then covered or closed, as appropriate.

The invention will now be further described by way of the following non-limiting examples which further illustrate the invention, and are not intended, nor should they be interpreted to, limit the scope of the invention.

### Examples

### Example 1

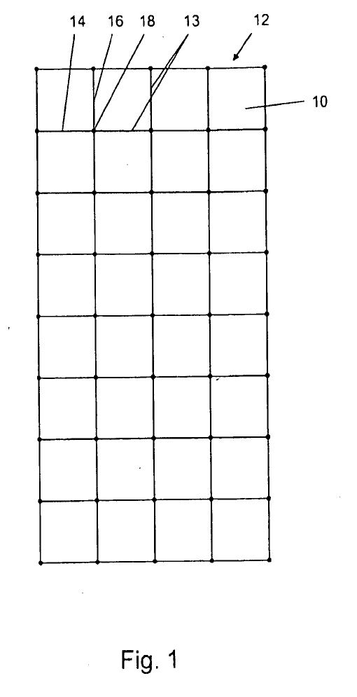

A composite material according to one embodiment of the invention was prepared. The composite material is demonstrated in FIG. 1, which shows a treated section 10 of acellular allograft or xenograft tissue which is generally rectangular in shape with a substantially planar surface having a dimension of about 3 cm to about 5 cm in width and about 6 cm to about 10 cm in length with a thickness of about 0.2 mm to about 0.8 mm. A reinforcing mesh 12 constructed of a multifilament polyester 13 with longitudinal strands 14 and transverse strands 16 are fused together at fuse points 18 to form a mesh of rectangular sections in an X and Y direction spaced about 1 cm on each side. The reinforcing mesh can have various designs such as squares, rectangles, ovals, circles, triangles, spirals and undulating but preferably has spaced dimensions ranging from about 0.1 cm to about 2.0 cm, preferably about 1.0 cm. The mesh is designed to last for at least about 1 month to about 6 months.

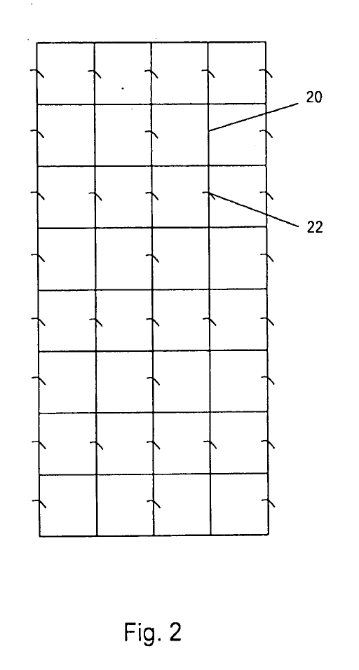

The fibers of the mesh 12 are made of a biocompatible material and may be, for example, knitted or weaved as shown in FIG. 2 which uses monofilament strands 20 held in place on the tissue 10 by sutures 22. It is also envisioned that staples can be used in the place of sutures to mount the strands to the tissue sheet.

### Example 2

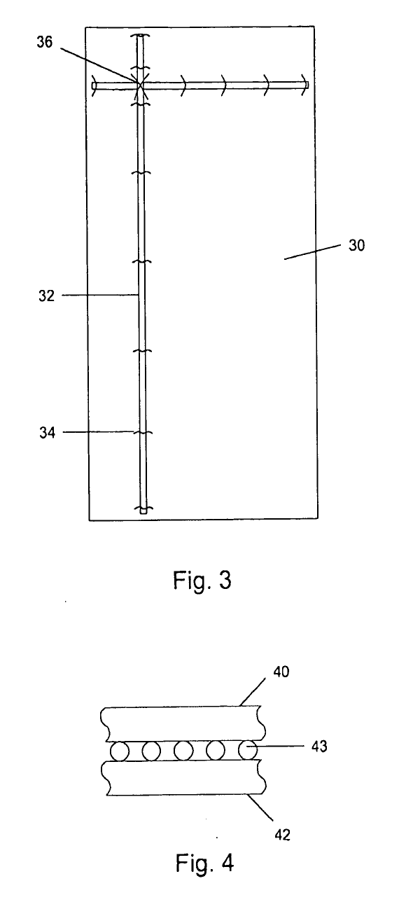

A composite material according to one embodiment of the invention was prepared. As is shown in FIG. 3, the composite material is an acellular sheet 30 reinforced by allograft or xenograft tendon fibers 32 which are stapled 34 onto the sheet and stapled 36 where the fibers intersect. The tendon fibers can be treated with anti-infectives to prevent infection as noted above.

### Example 3

A composite material according to one embodiment of the invention was prepared. As shown in FIG. 4, two acellular dermal sheets 40 and 42 are sandwiched around a fiber mesh 43 constructed of the same materials as described in Examples 1-3

While specific embodiments of the subject invention have been discussed, the above specification is illustrative and not restrictive. One skilled in the art will appreciate that numerous changes and modifications can be made to the invention, and that such changes and modifications can be made without departing from the spirit and scope of the invention. The full scope of the invention should be determined by reference to the claims, along with their full scope of equivalents, and the specification, along with such variations.

Each patent, patent application, and publication cited or described in the present application is hereby incorporated by reference in its entirety as if each individual patent, patent application, or publication was specifically and individually indicated to be incorporated by reference.

What is claimed is:

1. A composite material for use in a medical application, comprising at least one biological material and at least one reinforcement material.

2. The composite material of claim 1, wherein the biological material partially overlays the reinforcement material.

3. The composite material of claim 1, wherein the biological material overlays substantially all of the reinforcement material.

4. The composite material of claim 1, wherein the biological material is attached to the reinforcement material.

5. The composite material of claim 4, wherein the biological material is attached to the reinforcement material via an adhesive.

6. The composite material of claim 5, wherein the adhesive is selected from a group consisting of cyanoacrylate, glue, fibrin glue, fibrin, thrombin, plasma, and cellular-derived hemostatic agents.

7. The composite material of claim 4, wherein the biological material is attached to the reinforcement material via a mechanical agent.

8. The composite material of claim 7, wherein the mechanical agent is a suture or staple.

9. The composite material of claim 4, wherein fibers of the biological material are interwoven with fibers of the reinforcement material.

10. The composite material of claim 4, wherein the biological material and the reinforcement material are attached through physical crosslinking.

11. The composite material of claim 10, wherein the physical crosslinking involves dehydrothermal crosslinking, ultraviolet light, or heat.

12. The composite material of claim 4, wherein the biological material and the reinforcement material are attached through chemical crosslinking.

13. The composite material of claim 12, wherein the chemical crosslinking uses glutaraldehyde, formaldehyde, and carbodiimide.

14. The composite material of claim 4, wherein the biological material and the reinforcement material are attached by placement or precipitation of the biological material into the reinforcement material.

15. The composite material of claim 14, wherein the reinforcement material is swelled to allow placement or precipitation of the biological material.

16. The composite material of claim 14, wherein the reinforcement material comprises cavities to allow placement or precipitation of the biological material.

17. The composite material of claim 4, wherein the biological material and the reinforcement material are attached by a coating or spraying of the biological material onto the reinforcement material.

18. The composite material of claim 1, wherein the biological material is in a first layer, and the reinforcement material is in an adjacent second layer.

19. The composite material of claim 18, wherein the biological material is further in a third layer adjacent to the reinforcement material, wherein the second layer of reinforcement material is between the first layer of biological material and the third layer of biological material.

20. The composite material of claim 19, wherein the biological material of the first layer is the same as the biological material of the third layer.

21. The composite material of claim 19, wherein the biological material of the first layer is different than the biological material of the third layer.

22. The composite material of any one of claims 1-21, wherein the reinforcement material is in the form of a mesh.

23. The composite material of claim 22, wherein the mesh comprises a web, wherein the web is defined by a plurality of spaced apertures.

24. The composite material of claim 23, wherein the size of the spaced apertures are about 0.1 cm to about 2.0 cm.

25. The composite material of claim 1, wherein the biological material is selected from the group consisting of allograft, xenograft, autograft, and biologic matrix.

26. The composite material of claim 25, wherein the allograft, xenograft, or autograft is selected from the group consisting of dermis, fascia, fascia lata tendon, pericardia, ligament, and muscle.

27. The composite material of claim 1, wherein the biological material is acellular.

28. The composite material of claim 1, wherein the reinforcement material is non-biologic.

29. The composite material of claim 28, wherein the non-biologic reinforcement material is selected from the group consisting nylon, polyester, polypropylene, silk and cotton.

30. The composite material of claim 28, wherein the non-biologic reinforcement material is multifilament polyester strands.

31. The composite material of claim 28, wherein the non-biologic reinforcement material is monofilament strands.

32. The composite material of claim 1, wherein the reinforcement material is biologic.

33. The composite material of claim 32, wherein the biologic reinforcement material is selected from the group consisting of allograft, xenograft, autograft, and biologic matrix.

34. The composite material of claim 32, wherein the biologic reinforcement material is extracellular matrix proteins.

35. The composite material of claim 34, wherein the extracellular matrix proteins are selected from the group consisting of collagen, elastin, hyaluronic acid, and glycosaminoglycans.

36. The composite material of claim 32, wherein the biologic reinforcement material is connective tissue.

37. The composite material of claim 36, wherein the connective tissue is selected from the group consisting of tendon, ligament, and fascia.

38. The composite material of claim 32, wherein the biologic reinforcement material is bone or muscle.

39. The composite material of claim 1, wherein the reinforcement material can sustain a load of at least 10 Newtons.

40. A Method of preparing a composite material for use in a medical application, comprising:
(i) providing at least one biological material and at least one reinforcement material; and
(ii) overlaying the reinforcement material with the biological material.

41. A method of preparing a composite material for use in a medical application, comprising:
(i) providing at least one biological material and at least one reinforcement material; and
(ii) attaching the biological material to the reinforcement material.

42. The method if claim 41, wherein the biological material is attached to the reinforcement material via an adhesive.

43. The method of claim 42, wherein the adhesive is selected from a group consisting of cyanoacrylate, glue, fibrin glue, fibrin, thrombin, plasma, and cellular derived hemostatic agents.

44. The method of claim 41, wherein the biological material is attached to the reinforcement material via a mechanical agent.

45. The method of claim 44, wherein the mechanical agent is a suture or staple.

46. The method of claim 41, wherein fibers of the biological material are interwoven with fibers of the reinforcement material.

47. The composite material of claim 41, wherein the biological material and the reinforcement material are attached through physical crosslinking.

48. The composite material of claim 47, wherein the physical crosslinking involves dehydrothermal crosslinking, ultraviolet light, or heat.

49. The composite material of claim 41, wherein the biological material and the reinforcement material are attached through chemical crosslinking.

50. The composite material of claim 49, wherein the chemical crosslinking uses glutaraldehyde, formaldehyde, and carbodiimide.

51. The composite material of claim 41, wherein the biological material and the reinforcement material are attached by placement or precipitation of the biological material into the reinforcement material.

52. The composite material of claim 51, wherein the reinforcement material is swelled to allow placement or precipitation of the biological material.

53. The composite material of claim 51, wherein the reinforcement material comprises cavities to allow placement or precipitation of the biological material.

54. The composite material of claim 41, wherein the biological material and the reinforcement material are attached by a coating or spraying of the biological material onto the reinforcement material.

55. The method of claim 40 or 41, wherein the biological material is selected from the group consisting of allograft, xenograft, autograft, and biologic matrix.

56. The method of claim 55, wherein the allograft, xenograft, or autograft is selected from the group consisting of dermis, fascia, fascia lata tendon, pericardia, and ligament muscle.

57. The method of claim 40 or 41, wherein the biological material is acellular.

58. The method of claim 40 or 41, wherein the reinforcement material is non-biologic.

59. The method of claim 58, wherein the non-biologic reinforcement material is selected from the group consisting of nylon, polyester, polypropylene, silk and cotton.

60. The method of claim 58, wherein the non-biologic reinforcement material is multifilament polyester strands.

61. The method of claim 58, wherein the non-biologic reinforcement material is monofilament strands.

62. The method of claim 40 or 41, wherein the reinforcement material is biologic.

63. The method of claim 62, wherein the biologic reinforcement material is selected from the group consisting of allograft, xenograft, autograft, and biologic matrix.

64. The method of claim 62, wherein the biologic reinforcement material is extracellular matrix (ECM) proteins.

65. The method of claim 64, wherein the biologic reinforcement material is provided by precipitation of a particulate composition of ECM proteins.

66. The method of claim 64, wherein the biologic reinforcement material is provided by linking ECM proteins together to form larger molecules.

67. The method of claim 64, wherein the extracellular matrix proteins are selected from the group consisting of collagen, elastin, hyaluronic acid, and glycosaminoglycans.

68. The composite material of claim 62, wherein the biologic reinforcement material is connective tissue.

69. The composite material of claim 68, wherein the connective tissue is selected from the group consisting of tendon, ligament, and fascia.

70. The composite material of claim 62, wherein the biologic reinforcement material is bone or muscle.

71. The method of claim 62, wherein the biologic reinforcement material is provided by electrospinning biologic fibers.

72. The method of claim 62, wherein the biologic reinforcement material is provided by extruding biologic fibers.

73. The method of claim 62, wherein the biologic reinforcement material is provided by attaching nanoparticles to create larger ECM-based molecules, which forms the reinforcement material.

74. The method of claim 62, wherein the biologic reinforcement material is provided by using recombinant viral DNA to produce matrix from biologic material.

75. The method of claim 40 or 41, further comprising treating the biological material with at least one growth factor.

76. The method of claim 75, wherein the growth factor is selected from the group consisting of platelet-derived growth factor (PDGF), fibroblast growth factor (FGF 1-23) and variants thereof, transforming growth factor-beta (TGF-beta) and vascular endothelium growth factor (VEGF), Activin/TGF, steroids, and any combination thereof.

77. The method of claim 40 or 41, further comprising treating the reinforcement material with at least one anti-infectant.

78. The method of claim 77, wherein the anti-infectant is selected from the group consisting of anti-inflammatory agents, analgesic agents, local anesthetic agents, antispasmodic agents, and combinations thereof.

79. The method of claim 40 or 41, further comprising treating the composite material with one or more protease inhibitors.

80. The method of claim 79, wherein the protease inhibitor is selected from the group consisting of Aminoethylbenzenesulfonyl fluoride HCL, Aprotinin, Protease Inhibitor E-64, Leupeptin, Hemisulfate, EDTA, Disodium (0.025-0.10 um) and trypsin-like proteases, Pepstatin A (Aspartic Proteases), Marmistat (MMP2), and any combination thereof.

81. A method of repairing damaged tissue comprising implanting the composite material of claim 1 into the site of the damaged tissue.
